(19) United States
(12) Patent Application Publication
(10) Pub. No.: US 2018/0072632 A1
(43) Pub. Date: Mar. 15, 2018

(54) SILICA ENCAPSULATION OF UREOLYTIC BACTERIA FOR SELF-HEALING OF CEMENT-BASED COMPOSITES

(71) Applicant: Iowa State University Research Foundation, Inc., Ames, IA (US)

(72) Inventors: Kejin WANG, Ames, IA (US); Wenyu HUANG, Ames, IA (US); Gilson LOMBOY, Ames, IA (US); Yuchen PEI, Ames, IA (US); Zhiyuan QI, Ames, IA (US)

(21) Appl. No.: 15/703,682

(22) Filed: Sep. 13, 2017

Related U.S. Application Data
(60) Provisional application No. 62/394,565, filed on Sep. 14, 2016.

(51) Int. Cl. C04B 40/00; C04B 28/04; C04B 24/12; C04B 22/12; C04B 14/04; C12N 11/14

(52) U.S. Cl. CPC C04B 40/0039; C04B 28/04; C04B 24/126; C04B 2103/0001; C04B 14/04; C12N 11/14; C04B 22/124

(57) ABSTRACT

One aspect of the present invention is directed to a method of preparing encapsulated ureolytic cells. This method includes blending freeze dried ureolytic cells and an aqueous solution to form a base mixture; mixing the base mixture with a silicate-forming compound to form a blend comprising silica encapsulated ureolytic cells; and freeze drying the silica encapsulated ureolytic cells. The present invention also relates to a method of producing a self-healing concrete. This method comprises providing silica encapsulated freeze-dried ureolytic cells; mixing the silica encapsulated freeze-dried ureolytic cells with cement to form a mixture; and blending the mixture with a calcium salt and a urea solution to form a concrete mixture. Also disclosed are silica encapsulated ureolytic cells, a method of making a concrete form, and a cured concrete product.

This application claims the benefit of U.S. Provisional Patent Application Ser. No. 62/394,565, filed Sep. 14, 2016, which is hereby incorporated by reference in its entirety.

### FIELD OF THE INVENTION

The present application relates to the silica encapsulation of ureolytic bacteria for self-healing of cement-based composites.

### BACKGROUND OF THE INVENTION

Concrete is the most widely used construction material in the world. In 2014, China alone produced enough cement to make 330 billion cubic feet of concrete that can cover the entire island of Manhattan with a block 520 feet thick (Twombly, M., “Towering Above,” Explore Us, National Geographic (2016)). In the United States (US), the annual concrete production is over 500 million tons (Mehta, P. K., “Greening of the Concrete Industry for Sustainable Development,” Concrete International (2002)). Although concrete has many advantages and been widely used, cracking of this material is almost inevitable due to its low tensile strength and high brittleness. Cracks can result from various causes, such as external loads, differential settlement of structures, volume changes (expansion or contraction) due to temperature and moisture gradients (e.g., wet-dry and freeze-thaw cycling), and chemical reactions (sulfate attack, reinforcement corrosion, etc.). Cracks, even microcracks, play a critical role in the material transport properties. They permit air, water, and chemicals to penetrate into concrete, thus inducing various physical and chemical reactions, expediting the concrete deterioration, and shortening the structure service life. In transportation infrastructure, corrosion deterioration of reinforcing steel in bridge decks is often related to the penetration of deicing chemicals through microcracks into concrete. In buildings, cracks are primarily responsible for energy loss due to air infiltration. Detecting cracks, especially microcracks, within in-service structures is very challenging as cracks are often randomly distributed and invisible. Without timely treatment, microcracks tend to grow, permitting more fluids to get into concrete and causing more severe deterioration, thus requiring costly repair. It is estimated that the annual cost of concrete structure maintenance and repair is $18-21 billion in US alone (Emmons, P. H. and Sordyl, D. J., “The State of the Concrete Repair Industry, and a Vision of its Future,” Concrete Repair Bulletin 7-14 (2006)).

Typical crack repair uses cement grout, chemical sealants, or surface treatment agents. Not only are such crack repair techniques expensive and harmful to the environment, such a repair also requires prompt detection of cracks and estimation of the degree of damage in concrete. There is an urgent need for stopping concrete cracking using smart, eco-friendly, cost-effective, crack-controlling materials. Recently, the study of microorganism-based self-healing cement composites has shown promising results for meeting such a need. From the sustainability viewpoint, since microorganisms can be abundantly cultured, using microorganism-based cement to replace Portland cement and chemical binders would help reduce consumption of natural resources (i.e. raw materials used for producing cement and chemical binders) and emission of carbon dioxide.

Microorganism-based cement, also called biocement, is a mixture of enzyme or microbial biomass with inorganic chemicals through the microbiologically-induced calcium carbonate precipitation (MICP) process (Chu et al., “Biocement: Green Building- and Energy-Saving Material,” Advanced Materials Research v. 347-353:4051-4054 (2011)). A calcium-based biocement often consists of a biomass of urease producing bacteria (UPB), urea (CO(NH₂)₂), and a soluble calcium salt (e.g., CaCl₂). In the MICP process, UPB hydrolyzes urea and releases dissolved inorganic carbon (DIC) and ammonium (AMM) ions in the microenvironment of the bacteria (FIG. 1A). Calcium ions, from the calcium salt, are attracted to the bacterial cell wall due to the negative charge of the latter. As AMM ions increases the pH of the system increases, thus promoting microbial deposition or precipitation of calcium carbonate (CaCO₃) on the cell wall (FIG. 1B and Eq. 1) (Hammes et al., “Key Roles of pH and Calcium Metabolism in Microbial Carbonate Precipitation,” Rev. Environ. Sci. Biotechnol 1:3-7 (2002)).

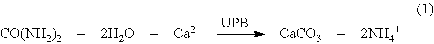

Such biogenic-CaCO₃ can bond loose particles together and clog and seal fine pores and cracks (FIG. 2). Research has also found that having lower viscosity, biocement grout is easy to penetrate into very fine spaces in porous materials (Plank, J., “Application of Biopolymers and Other Biotechnological Products in Building Material,” Appl Microbiol Biotechnol 66:1-9 (2004)). Due to the environmental and economic benefits, MICP is increasingly used for stone surface protection, crack sealing, sand cementation, and soil consolidation. However, the above-described biocement can only fix cracks in damaged materials manually (by injecting, spraying, or soaking), rather than intrinsically or naturally mending the growing cracks in construction materials.

Self-healing material with a microencapsulated healing agent was reported in 2001 (White et al., “Autonomic Healing of Polymer Composites,” Nature 409:794-797 (2001)). As shown in FIG. 3, when a material containing a micro-encapsulated healing agent cracks, the crack propagation will break the shells of the embedded capsules and release the healing agent, which in turn fills and automatically mends the cracks. Although micro-encapsulation has been rapidly used in polymers and composites, its research and application for cement-based materials started very recently. Among the recent studies, a few self-healing cement-based composites have been developed based on the MICP concept, where porous materials, such as tubular fibers (Yang et al., “A Self-healing Cementitious Composite Using Oil Core/Silica Gel Shell Microcapsules,” Cement and Concrete Composites 33(4):506-512 (2011)), expanded clay particles (Wiktor et al., “Quantification of Crack-healing in Novel Bacteria-based Self-healing Concrete,” Cement and Concrete Composites 33:763-770 (2011)), and glass capillaries (Wang et al., “Use of Silica Gel or Polyurethane Immobilized Bacteria for Self-healing Concrete,” Construction and Building Materials 26(1):532-540 (2012)), are often used as a bacteria carrier. Application of such microorganism-based encapsulation has been reported in remediation of cracks (Achal et al., “Corrosion Prevention of Reinforced Concrete With Microbial Calcite Precipitation,” ACI Materials Journal 157-163 (2012); Van Tittelboom et al., “Use of Bacteria to Repair Cracks in Concrete,” Cement and Concrete Research 40(1):157-166 (2010); Bang et al., “Microbial Calcite, a Bio-based Smart Nanomaterial in Concrete Remediation,” International Journal of Smart and Nano Materials 1(1):28-39 (2010); Ramachandran et al., “Remediation of Concrete Using Microorganisms,” ACI Materials Journal 98:3-9 (2001); and De Muynck et al., “Bacterial Carbonate Precipitation as an Alternative Surface Treatment for Concrete,” Construction and Building Materials 22:875-885 (2008)), soil improvement (Al-Thawadi, “Ureolytic Bacteria and Calcium Carbonate Formation as a Mechanism of Strength Enhancement of Sand,” J. Adv. Science and Eng. Research 1:98-114 (2011); Barkouki et al., “Forward and Inverse Bio-mediated Modeling of Microbially Induced Calcite Precipitation in Half-meter Column Experiments,” Transport in Porous Media 90:23-39 (2011); Chou et al., “Biocalcification of Sand Through Ureolysis,” J Geotech. Geoenviron. Eng. 127(12):1179-1189 (2011); DeJong et al., “Microbial Induced Cementation to Control Sand Response to Undrained Shear,” J. Geotech. Geoenviron. Eng. 132(11):1381-1392 (2006); DeJong et al., “Bio-mediated Soil Improvement,” Ecol. Eng 36(2):197-210 (2010); Rong et al., “A Cementation Method of Loose Particles Based on Microbe-based Cement,” Science China: Technological Sciences 54(7):1722-1729 (2011); Van Paassen et al., “Quantifying Biomediated Ground Improvement by Ureolysis: Large-scale Biogrout Experiment,” J. Geotech. Geoenviron. Eng 136(12):1721-1728 (2010); and Whiffin et al., “Microbial Carbonate Precipitation as a Soil Improvement Technique,” Geomicrobiol. J. 24:417-423 (2007)), and sequestration of radionuclides and heavy metals (Fujita et al., “Strontium Incorporation Into Calcite Generated by Bacterial Ureolysis,” Geochim. Cosmochim. Acta 68(15):3261-3270 (2004); Curti, E, “Coprecipitation of Radionuclides with Calcite: Estimation of Partition Coefficients Based on a Review of Laboratory Investigations and Geochemical Data,” Appl. Geochem 14:433-445 (1999); Zachara et al., “Sorption of Divalent Metals on Calcite,” Geochim. Cosmochim. Acta 55:1549-1562 (1991); Pingitore et al., “The Coprecipitation of Sr2+ and Calcite at 25 Degrees C. and 1 atm,” Geochim. Cosmochim. Acta 50(10):2195-2203 (1986)).

These studies have clearly demonstrated that the concept of using MICP for self-healing cracks in concrete is promising. However, the efficiency of the bacteria carriers and their effects on the properties of cement-based materials haven't been researched yet. Most bacteria carriers have a relatively large size and often carry clusters of bacteria which have quite different physical and surface chemical properties from cement-based materials. As a result, they may adversely affect the homogeneity of cement-based composites, form weak spots, damage the material integrity, and reduce the material strength.

The present invention is directed to overcoming the above-noted deficiencies in the art.

### SUMMARY OF THE INVENTION

One aspect of the present invention is directed to a method of preparing encapsulated ureolytic cells. This method includes blending freeze dried ureolytic cells and an aqueous solution to form a base mixture; mixing the base mixture with a silicate-forming compound to form a blend comprising silica encapsulated ureolytic cells; and freeze drying the silica encapsulated ureolytic cells.

The present invention also relates to freeze-dried ureolytic cells encapsulated with silica.

Another aspect of the present invention pertains to a method of producing a self-healing concrete. This method comprises providing silica encapsulated freeze-dried ureolytic cells; mixing the silica encapsulated freeze-dried ureolytic cells with cement to form a mixture; and blending the mixture with a calcium salt and a urea solution to form a concrete mixture.

A further aspect of the present invention is directed to a concrete product mixture comprising: freeze-dried ureolytic cells encapsulated with silica; concrete; urea solution; and calcium salt.

An additional aspect of the present invention is directed to a method of making a concrete form by forming the concrete product mixture into a desired shape and curing the formed concrete. The resulting cured concrete product is yet a further embodiment of the present application.

Using microorganism-based microcapsules for self-healing concrete has many challenges: (1) encapsulation serves as protection for the bacteria from being crushed during concrete mixing and casting and from being attacked by the high alkalinity of concrete pore water; (2) the microcapsules should be easily dispersed and well distributed to keep the homogeneity of the cement-based system; (3) the shells of the microcapsules should be water-tight to keep the freeze-dried bacteria inside in a dormant condition which retains their viability; (4) when concrete cracks, the shells of the microcapsules should fracture and release the bacteria; (5) the bacteria released should be able to revive at their exposure environment conditions to participate in the MICP process; and (6) the rate of CaCO₃ precipitation has to be appropriate to seal microcracks in a timely fashion.

To meet the above-mentioned challenges, selection of bacteria type, materials used for encapsulation, and encapsulation methods are important. These objectives are achieved by a method for encapsulation of ureolytic bacteria in accordance with the present invention. The method involves mixing the bacteria with urea to facilitate the reaction of an organosilicate (e.g., tetraethyl orthosilicate (TEOS)) and produce a silica shell to encapsulate the bacteria. Since Portland cement consists mainly of hydraulic calcium silicates, these micro-sized silica capsules interact with a cement-based system very well.

The examples of the present application (see FIG. 4) show that encapsulated bacteria were able to sustain a standard cement paste mixing procedure. Through mixing, the microcapsules were well distributed in the paste. The capsules were broken easily as the hardened cement paste fractured. The encapsulated bacteria released from the microcapsules were able to revive and undergo the MICP process in a urea-CaCl₂ solution. The precipitation of CaCO₃ observed (also confirmed by thermogravimetric analysis (TGA)) will bond loose particles and seal cracks as well as fine pores in concrete.

### BRIEF DESCRIPTION OF THE DRAWINGS

FIGS. 1A-1B illustrate ureolytic induced carbonate precipitation (De Muynck et al., “Microbial Carbonate Precipitation in Construction Materials: A Review,” Ecological Engineering 36:118-13 (2010), which is hereby incorporated by reference in its entirety).

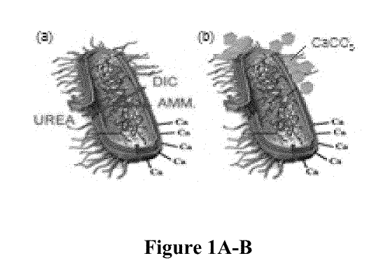

FIG. 2 shows concrete crack sealed with biocement (Courtesy of Delft University of Technology).

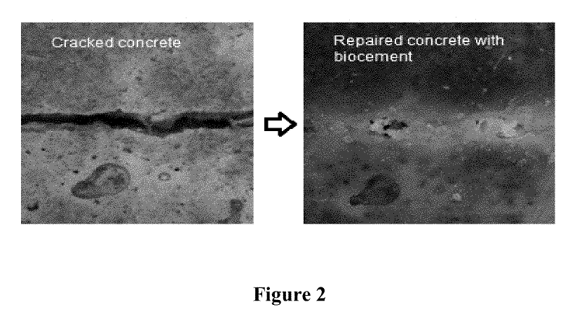

FIGS. 3A-C show the self-healing concept (White et al., “Autonomic Healing of Polymer Composite,” Nature 409:794-797 (2001), which is hereby incorporated by reference in its entirety).

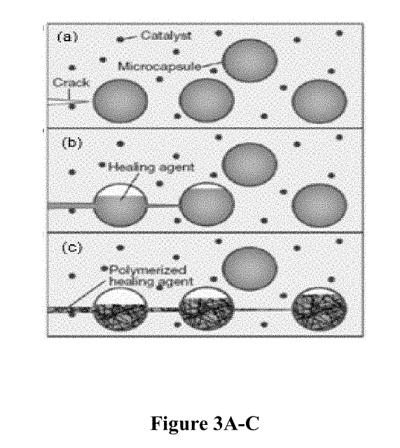

FIGS. 4A-D show the viability of encapsulated UPB in a cement paste produced in a lab condition (Note: The results indicate that after encapsulation, mixing, and fracturing, the UPB was still able to mediate precipitation of CaCO₃).

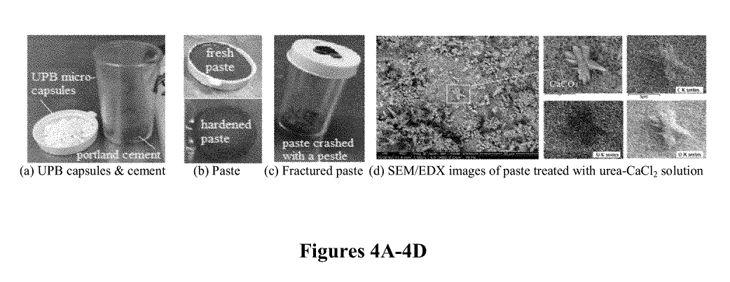

FIG. 5 shows encapsulated bacteria (in a small container) and Portland cement (in a vial) prior to mixing of cement pastes.

FIG. 6 shows cement paste with encapsulated bacteria in a mold, before sealing.

FIG. 7 shows hardened cement paste with encapsulated bacteria before crushing.

FIG. 8 shows crushed hardened cement paste with encapsulated bacteria.

FIG. 9 shows the Scanning Electron Microscopy (SEM) image of the precipitate found in a vial after treatment of the crushed encapsulated bacteria.

FIG. 10 shows the surface of crushed cement paste with encapsulated bacteria after treatment with a solution of urea and calcium chloride.

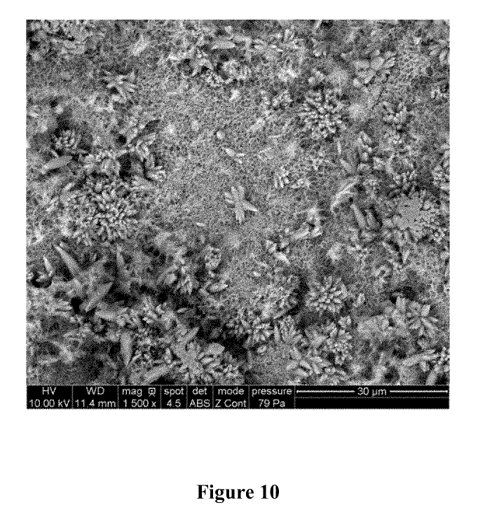

FIG. 11 shows calcium carbonate formation on the surface of the treated crushed cement paste.

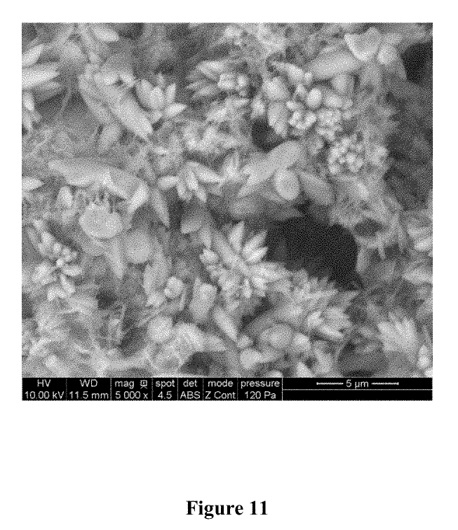

FIG. 12 shows the partial composition of formation on the surface of the treated crushed cement paste.

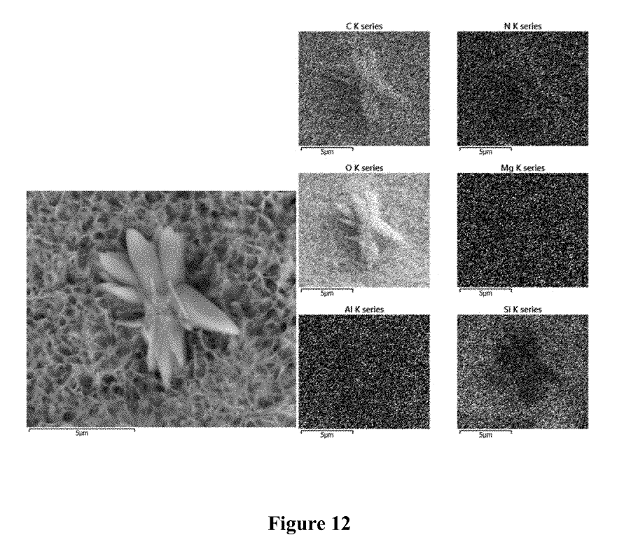

FIG. 13 shows Thermo-Gravimetric Analysis (TGA) results of treated (red) and untreated (blue) crushed cement paste.

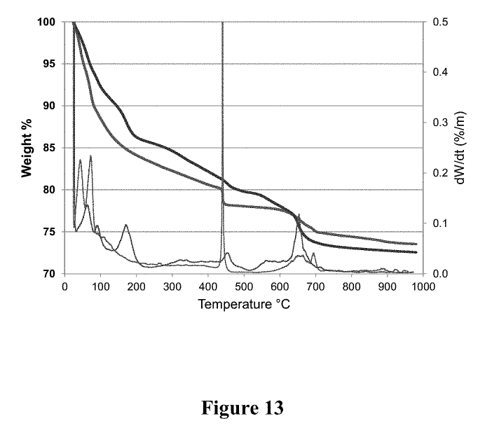

FIG. 14 shows Diffuse Reflectance Infrared Fourier Transform Spectroscopy (DRIFTS) spectra of sample 3 and 4 in the bacteria viability test of encapsulation.

FIG. 15 shows powder X-Ray diffraction (PXRD) patterns of sample 3 and 4 in the bacteria viability test of encapsulation.

### DETAILED DESCRIPTION OF THE INVENTION

One aspect of the present invention is directed to a method of preparing encapsulated ureolytic cells. This method includes blending freeze dried ureolytic cells and an aqueous solution to form a base mixture; mixing the base mixture with a silicate-forming compound to form a blend comprising silica encapsulated ureolytic cells; and freeze drying the silica encapsulated ureolytic cells.

As used herein, the term “ureolytic bacteria” refers to bacteria which hydrolyze urea to generate adenosine triphosphate (ATP) by the efflux of ammonium ions through ATP-synthase, producing carbonate.

The ureolytic cells may be Sporosacina pateurii, Sporsacina ureae, Bacillus sphaericus, Bacillus pseudofirmus, Bacillus cohnii, or Bacillus alkalinitrilicus.

The base mixture contains a base precursor. The base precursor can be added separately or produced by cells. As used herein, the term “base precursor” refers to a substance from which a base is produced. Exemplary base precursors include, but are not limited to, urea, thiourea, and dimethylurea.

The blending step can be carried out at a temperature of 0 to 100° C. The blending step involves the hydration of the freeze dried ureolytic cells and initiates ureolysis.

The mixing step can be carried out at a temperature of 20 to 50° C. As a result of such mixing, the encapsulation of ureolytic cells by metabolic ureolysis is induced.

The silicate-forming compound is an organosilicate compound such as, tetraethyl orthosilicate, tetramethyl orthosilicate, tetraprophy orthosilicate, and tetrabutyl orthosilicate.

The method further includes washing the mixture and recovering the encapsulated cells prior to said freeze drying. Washing can be achieved by suspending the mixture in an aqueous solution. Recovering can be carried out by precipitation, filtration, or centrifugation.

As used herein the term “freeze drying” or “freeze dried” refers to the removal of water from a frozen material using sublimation. Exemplary methods of freeze-drying bacterial cells are described in detail in Franks et al., “Effective Freeze-Drying: a Combination of Physics, Chemistry, Engineering and Economics.” Proc. Inst. Refrigeration 91: 32-39 (1994), which is hereby incorporated by reference in its entirety.

In some embodiments the silica encapsulated ureolytic cells may be freeze dried by immersing the silica encapsulated ureolytic cells into liquid nitrogen.

In other embodiments the silica encapsulated ureolytic cells may be freeze dried using a freezing step and a drying step.

The freezing step may include any method that is suitable for the freezing of the encapsulated ureolytic cells. For example, freezing may be carried out by placing the material in a freeze-drying flask and rotating the flask in a bath, also known as a shell freezer, which is cooled by, for example, mechanical refrigeration, by a mixture of dry ice with an alcohol such as methanol or ethanol, or by liquid nitrogen. Alternatively, freezing may be carried out using a commercially available freeze-drying apparatus or a temperature controlled freeze-drying machine. The encapsulated cells are rapidly frozen in order to avoid the formation of ice crystals. Freezing temperatures may range between −50° C. and −80° C.

The drying step may involve lowering the pressure using a vacuum (typically to the range of a few millibars) and applying sufficient heat to the material for the water to sublimate. The amount of heat necessary can be calculated using the sublimating molecules' latent heat of sublimation.

The present invention also relates to freeze-dried ureolytic cells encapsulated with silica.

The freeze-dried ureolytic cells can be Sporosacina pateurii, Sporsacina ureae, Bacillus sphaericus, Bacillus pseudofirmus, Bacillus cohnii, or Bacillus alkalinitrilicus.

Another aspect of the present invention pertains to a method of producing a self-healing concrete. This method comprises providing silica encapsulated freeze-dried ureolytic cells; mixing the silica encapsulated freeze-dried ureolytic cells with cement to form a mixture; and blending the mixture with a calcium salt and a urea solution to form a concrete mixture.

As used herein the term “cement” refers to an initially dry substance that develops compressive strength or sets in the presence of water. Suitable cements are known in the art and include, but are not limited to, Portland cement, blended Portland cement, masonry cement, expansive cement, rapid setting and hardening cement, calcium aluminate cement, calcium sulfoaluminate cement, pozzolan-lime cement, slag-lime cement, supersulfated cement, natural cement, geopolymer cement, magnesium-based cement, magnesium silicate cement, magnesium phosphate cement, calcium silicate cement, aluminum silicate cement, potassium silicate cement, sodium silicate cement, lithium silicate cement, and any mixtures and combinations thereof.

As used herein the term “concrete” refers to a material made by mixing a cementing material, an aggregate, and sufficient water to set and bind the mixture. Exemplary aggregates include, but are not limited to, sand, gravel, and crushed stone.

As used herein the term “self-healing concrete” refers to a concrete material capable of microbiologically-induced calcium carbonate precipitation (MICP). In some embodiments, the self-healing concrete comprises the silica encapsulated freeze-dried ureolytic cells, urea, and a calcium salt.

The self-healing concrete may optionally include fibers. Suitable fibers include, but are not limited to glass fibers, silicon carbide, aramid fibers, polyester, carbon fibers, composite fibers, fiberglass, steel fibers and combinations thereof, as well as, fabric containing the above-mentioned fibers, and fabric containing combinations of the above-mentioned fibers. Fibers may be used in a mesh structure, intertwined, interwoven, and oriented in any desirable direction. Exemplary fibers include, but are not limited to, MeC-GRID®, C-GRID®, KEVLAR®, TWARON®, DACRON®, and VECTRAN®.

In some embodiments, the fibers comprise at least 0.1, 0.5, 1, or 2 volume percent of the concrete composition. In other embodiments, the fibers provide up to 10, 9, 8, 7, 6, 5, 4, or 3 volume percent of the concrete composition. The amount of fibers may be adjusted to provide desired properties to the concrete composition and can be any value or range between any of the values recited above.

The self-healing concrete may optionally contain one or more additives, which include, but are not limited to, air entraining agents, anti-foam agents, water-proofing agents, dispersing agents, set-accelerators, set-retarders, plasticizing agents, water reducers, bonding agents, freezing point decreasing agents, adhesiveness-improving agents, and colorants. The additives are typically present at less than one percent by weight with respect to total weight of the composition, but can be present at from 0.1 to 3 weight percent.

Suitable air entraining agents are well known in the art and include, but are not limited to, foaming agents and resins.

Suitable dispersing agents or plasticizers include, but are not limited to, hexametaphosphate, tripolyphosphate, polynaphthalene sulphonate, sulphonated polyamine, and combinations thereof.

Suitable plasticizing agents that can be used in the invention include, but are not limited to, polyhydroxycarboxylic acids or salts thereof, polycarboxylates or salts thereof; lignosulfonates, polyethylene glycols, and combinations thereof.

Suitable freezing point decreasing agents that can be used in the invention include, but are not limited to, ethyl alcohol, calcium chloride, potassium chloride, and combinations thereof.

Suitable adhesiveness-improving agents that can be used in the invention include, but are not limited to, polyvinyl acetate, styrene-butadiene, homopolymers and copolymers of (meth)acrylate esters, and combinations thereof.

Suitable water-repellent or water-proofing agents that can be used in the invention include, but are not limited to fatty acids (such as, stearic acid or oleic acid), lower alkyl fatty acid esters (such as, butyl stearate), fatty acid salts (such as, calcium or aluminum stearate), silicones, wax emulsions, hydrocarbon resins, bitumen, fats and oils, silicones, paraffins, asphalt, waxes, and combinations thereof.

Examples of suitable bonding agents include materials that can be either inorganic or organic and are soft and workable when fresh but set to form a hard, infusible solid on curing, either by hydraulic action or by chemical crosslinking. Non-limiting examples of such materials can include organic materials, such as, rubber, polyvinyl chloride, polyvinyl acetate, acrylics, styrene butadiene copolymers, and various powdered polymers.

The cells are Sporosacina pateurii, Sporsacina ureae, Bacillus sphaericus, Bacillus pseudofirmus, Bacillus cohnii, or Bacillus alkalinitrilicus.

The calcium salt is calcium chloride, calcium acetate, calcium bromide, calcium lactate, calcium citrate, calcium nitrate, or calcium gluconate.

The mixing and blending steps may be carried out as described above.

A further aspect of the present invention is directed to a concrete product mixture comprising: freeze-dried ureolytic cells encapsulated with silica; concrete; urea solution; and calcium salt. In one example, the concrete product mixture comprises 1×10⁷ to 1×10⁹ cells per cm³. The concrete product mixture can include 0.1 to 10M urea. In some embodiments, the concrete product mixture comprises 0.1 to 5M calcium salt.

In the concrete product, the ureolytic cells are Sporosacina pateurii, Sporsacina ureae, Bacillus sphaericus, Bacillus pseudofirmus, Bacillus cohnii, or Bacillus alkalinitrilicus.

The calcium salt is calcium chloride, calcium acetate, calcium bromide, calcium lactate, calcium citrate, calcium nitrate, and calcium gluconate.

Also disclosed is a method of making a concrete form. This involves forming the concrete product into a desired shape and curing the formed concrete.

Forming the concrete product mixture may involve providing a mold or frame and transferring the concrete product mixture to the mold or frame using a pump, by pouring, or by any other means known in the art.

The formed concrete product mixture may be compacted to remove large air voids developed during the transferring step using vibration, roller compacting, or through the use of chemical admixtures.

The forming step is carried out at 10 to 32° C.

As used herein the term “curing” refers to the maintenance of a satisfactory moisture content and temperature in concrete for a period of time immediately following the forming the concrete product into a desired shape. Proper curing of concrete influences the properties of the hardened concrete, including durability, strength, watertightness, abrasion resistance, volume stability, and resistance to freeze-thawing.

Methods of curing concrete are well known in the art and include, but are not limited to, ponding or immersion, spraying or fogging, covering concrete with moisture-saturated fabrics, impervious paper, plastic sheeting materials, treatments with liquid membrane-forming compounds, internal moist curing, steam curing, use of insulating blankets or covers, electrical curing, hot oil curing, or microwave curing.

Suitable membrane forming compounds include, but are not limited to, waxes, resins, and chlorinated rubber.

The curing step is carried out at 16 to 27° C.

The ureolytic cells are Sporosacina pateurii, Sporsacina ureae, Bacillus sphaericus, Bacillus pseudofirmus, Bacillus cohnii, or Bacillus alkalinitrilicus.

The cured concrete product of this method is another aspect of the present invention.

### Examples

### Example 1—Materials and Methods

### Materials

Bacillus pasteurii (DSM 33) encapsulated with silica was tested to determine its viability after encapsulation, and after mixing and breaking of the cement paste. The cement used was Type I Portland cement. The water-to-cement ratio of the cement paste was 0.4.

When the bacteria were tested for viability, it was treated with a solution made of 2 M urea (CO(NH₂)₂) to 1M calcium chloride. The solution was used to determine whether the ureolytic bacterial was still capable of producing calcium carbonate.

### Viability Testing of Bacteria of Encapsulation

To determine the viability of the bacteria after encapsulation, 0.1 grams of encapsulated bacteria was placed in a mortar and broken by grinding using a pestle. The encapsulated bacteria were then taken out of the mortar and placed in a vial. The solution (urea with calcium chloride) was then poured inside the vial with the broken capsules with exposed bacteria. The vial was sealed for 7 days, and was shaken once a day to agitate the mixture. After 7 days, the content of the vial was poured out, washed, and dried. Precipitates in the wall of the vial remain after drying. The precipitates were viewed in a scanning electron microscope (SEM).

### Viability Testing of Bacteria in Cement Paste

To determine the viability of the bacteria for use with cement-based materials, 0.5 grams of the encapsulated bacteria was mixed with 4 grams of Portland cement and 1.6 grams of water. The mixture was placed in a mold and sealed with plastic for 14 days in 70 degrees Celsius. The hardened paste was then crushed to expose the bacteria, which also simulates concrete damage.

Half of the crushed sample was treated with the solution for 7 days. The treated sample was viewed under a SEM to find microbiologically induced calcium carbonate. Both treated and untreated crushed samples were subjected to thermo-gravimetric analysis (TGA) to confirm whether calcium carbonates were formed microbiologically.

### Viability Testing of Bacteria of Encapsulation

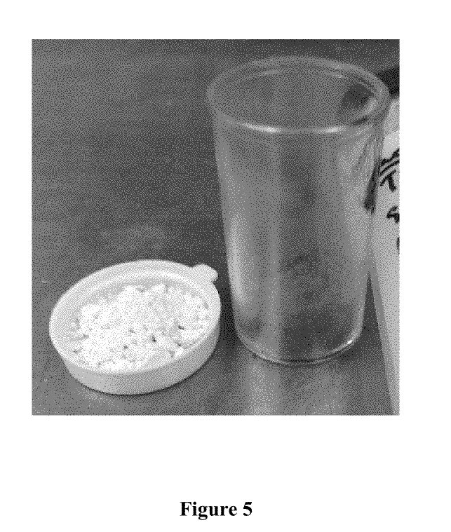

The SEM image of the precipitates remaining in the vial after treatment is shown in FIG. 5. The image shows both shapes formed by the bacteria and possible empty shell of capsules.

### Viability Testing of Bacteria in Cement Paste

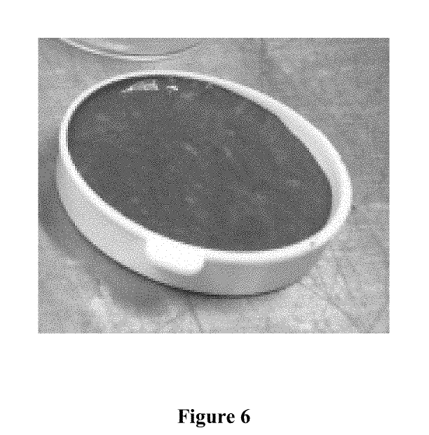

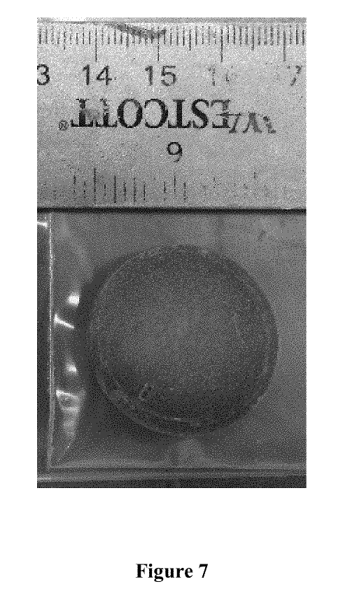

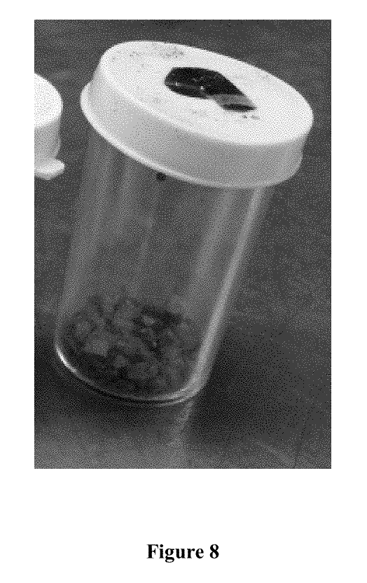

The SEM image of the surface of the treated crushed cement paste with bacteria is shown in FIG. 6. A magnified view of the calcium carbonate formation is shown in FIG. 7. The calcium carbonate formed have a scalenohedral form. The composition of the formation shown in FIG. 8 shows the presence of carbon and oxygen, with the absence of silica and alumina; indicating the presence of calcium carbonate and absence of cement hydration product.

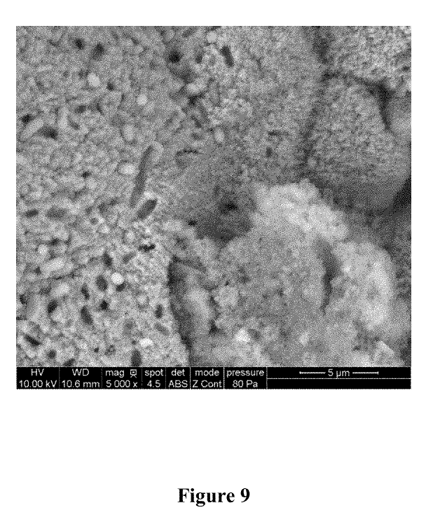

Further verification of the microbiological formation of calcium carbonate using TGA indicates a slightly greater amount of calcium carbonate in the treated crushed paste compared to the untreated crushed paste, as shown in FIG. 9. The measured calcium carbonate from the treated sample is 4.2% and the untreated sample only had 3.1%. The values are close, because the measurement was based on the total volume of the crushed paste, while the additional calcium carbonate was formed only on the surface of the treated sample. Calcium carbonate from a treated and untreated cement paste can also come from carbonation during the processing of the sample.

### Example 2—Encapsulation of Bacteria with SiO₂ Shell Induced by Metabolic Ureolysis (Method A)

Typically, 200 milligrams urea pellets were dissolved in 10 mL water. 100 mg bacteria solid was then mixed well with the urea solution. The bacteria-urea mixture was placed into a 30° C. oil bath with addition of TEOS for 24 hrs. After 24 hrs cultivation, the brown paste of bacteria and silica was collected by centrifugation under 7000 rpm for 10 min and washed with water for 3 times. The brown paste was then immersed into liquid nitrogen and freeze-dried. The dried white solid was stored in a −4° C. refrigerator for future use.

### Example 3—Synthesis Conditions

In method A, urea-bacteria solution had a neutral pH of around 7 before the 30° C. cultivation. After cultivation for 24 hrs, the solution turned into a white brown slime with a pH around 8-9, indicating that ureolysis of bacteria using urea can produce NH₃, which can basify the solution. The basic environment can facilitate the hydrolysis of TEOS to induce the silica encapsulation. The amount of added TEOS was turned to change the bacteria loadings, and the freeze-dry time was also tested as shown in Table 1.

TABLE 1
Synthesis conditions of method A.

| | Final mass/g | TEOS/mL | Drying time/h |
|---|---|---|---|
| 1 * | 0.1 | 1 | 3 |
| 2 * | 1.9 | 2 | 3 |
| 3 ^{†} | 0.29 | 1 | 2 |
| | | | 120 |
| 4 ^{†} | 0.55 | 2 | 2 |
| | | | 120 |

* using filtration which inducing loss;
^{†} after synthesis, the bacteria-silica paste was separated in half, one for 3 hr dry, the other partition was dried for 5 days, and the final mass is the overall mass of two halves.

Compared to items 2 and 4, it is clearly seen that the bacteria-silica solid, after 2 hrs drying, still has H₂O. From item 3 and 4, the TEOS addition is almost linear to the final mass of bacteria-silica, indicating a similar hydrolysis mechanism of TEOS.

### Example 4—Identification of the CaCO₃ Powder in the Bacteria Viability Test of Encapsulation

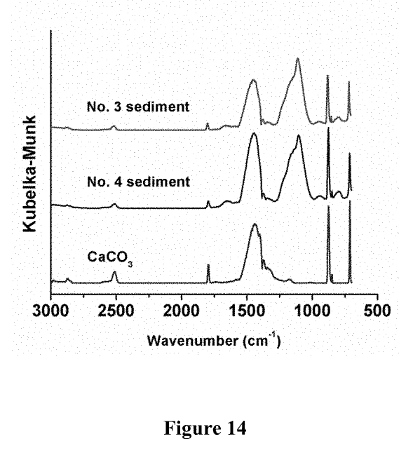

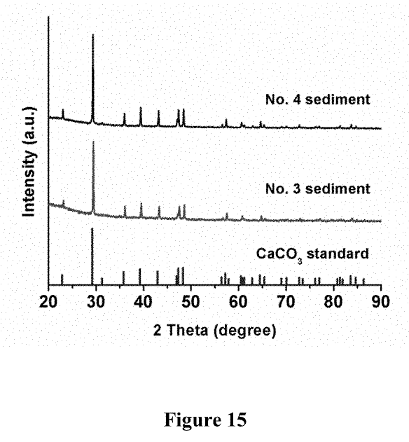

FIGS. 14 and 15 show the DRIFTS spectra and PXRD patterns of the white powder scratched from the plastic wafer in the bacteria viability test of encapsulation. Samples 3 and 4 are from method A using 1 mL TEOS and 2 mL TEOS, respectively. Both samples 3 and 4 in FIG. 14 show similar peaks of the CO₃²⁻ vibrations for CaCO₃ at around 710, 870, 1080, and 1430 cm⁻¹ which are characteristic bands of CO₃²⁻ (Table 2).

TABLE 2
Standard CO₃²⁻ vibrations.

| v, CO₃²⁻ | reference wavenumber (cm⁻¹) * |
|---|---|
| v₂, out-of-plane bend | 712 |
| V₃, asymmetric stretch | 874 |
| v₁, symmetric stretch | 1087 |
| v₄, in-plane bend | 1435 |

* H. A. Al-Hosney et al., Phys. Chem. Chem. Phys., 7: 1266-1267 (2005), which is hereby incorporated by reference in its entirety.

There is a broad peak at around of 1300 cm⁻¹ in both samples 3 and 4, which can be identified as C—H vibrations due to the organic remnants. PXRD patterns (FIG. 15) of samples 3 and 4 are also in great agreement with the pattern of standard calcite. DRIFTS and PXRD show that the white powder from the bacteria viability test of encapsulation is indeed CaCO₃ due to the ureolysis of bacteria.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of preparing encapsulated ureolytic cells, said method comprising:
blending freeze dried ureolytic cells and an aqueous solution to form a base mixture;
mixing the base mixture with a silicate-forming compound to form a blend comprising silica encapsulated ureolytic cells; and
freeze drying the silica encapsulated ureolytic cells.

2. The method of claim 1, wherein the solution contains a base precursor.

3. The method of claim 2, wherein the base precursor is urea.

4. The method of claim 1, wherein the ureolytic cells are selected from the group consisting of Sporosacina pateurii, Sporsacina ureae, Bacillus sphaericus, Bacillus pseudofirmus, Bacillus cohnii, and Bacillus alkalinitrilicus.

5. The method of claim 1, wherein the silicate-forming compound is an organosilicate compound.

6. The method of claim 5, wherein the organosilicate compound is selected from the group consisting of tetraethyl orthosilicate, tetramethyl orthosilicate, tetraprophy orthosilicate, and tetrabutyl orthosilicate.

7. The method of claim 1 further comprising:
washing the mixture and
recovering the encapsulated cells prior to said freeze drying.

8. The method of claim 7, wherein said recovering is carried out by precipitation or centrifugation.

9. The method of claim 1, wherein said blending is carried out at a temperature of 0 to 100° C.

10. The method of claim 1, wherein said mixing is carried out at a temperature of 20 to 50° C.

11. The silica encapsulated ureolytic cells produced by the method of claim 1.

12. Freeze-dried ureolytic cells encapsulated with silica.

13. The freeze-dried ureolytic cells of claim 12, wherein the cells are selected from the group consisting of Sporosacina pateurii, Sporsacina ureae, Bacillus sphaericus, Bacillus pseudofirmus, Bacillus cohnii, and Bacillus alkalinitrilicus.

14. A method of producing a self-healing concrete comprising:
providing silica encapsulated freeze-dried ureolytic cells;
mixing the silica encapsulated freeze-dried ureolytic cells with cement to form a mixture; and
blending the mixture with a calcium salt and a urea solution to form a concrete mixture.

15. The method of claim 14, wherein the ureolytic cells are selected from the group consisting of Sporosacina pateurii, Sporsacina ureae, Bacillus sphaericus, Bacillus pseudofirmus, Bacillus cohnii, and Bacillus alkalinitrilicus.

16. The method of claim 14, wherein the calcium salt is selected from the group consisting of calcium chloride, calcium acetate, calcium bromide, calcium lactate, calcium citrate, calcium nitrate, and calcium gluconate.

17. The method of claim 14, wherein said mixing is carried out at 10 to 32° C.

18. The method of claim 14, wherein said blending is carried out at 10 to 32° C.

19. The concrete product produced by the method of claim 14.

20. A concrete product mixture comprising:
freeze-dried ureolytic cells encapsulated with silica;
concrete;
urea solution; and
calcium salt.

21. The concrete product of claim 20, wherein the ureolytic cells are selected from the group consisting of Sporosacina pateurii, Sporsacina ureae, Bacillus sphaericus, Bacillus pseudofirmus, Bacillus cohnii, and Bacillus alkalinitrilicus.

22. The concrete product of claim 20, wherein the calcium salt is selected from the group consisting of calcium chloride, calcium acetate, calcium bromide, calcium lactate, calcium citrate, calcium nitrate, and calcium gluconate.

23. A method of making a concrete form comprising:
forming the concrete product of claim 20 into a desired shape and
curing the formed concrete.

24. The method of claim 23, wherein the ureolytic cells are selected from the group consisting of Sporosacina pateurii, Sporsacina ureae, Bacillus sphaericus, Bacillus pseudofirmus, Bacillus cohnii, and Bacillus alkalinitrilicus.

25. The method of claim 23, wherein said forming is carried out at 10 to 32° C.

26. The method of claim 23, wherein said curing is carried out at 16 to 27° C.

27. The cured concrete product of claim 23.
